(19) United States
(12) Patent Application Publication
(10) Pub. No.: US 2025/0005986 A1
(43) Pub. Date: Jan. 2, 2025

(54) PASSAGE PERMIT DEVICE, SYSTEM, METHOD, AND NON-TRANSITORY COMPUTER READABLE MEDIUM STORING PROGRAM

(71) Applicant: NEC Corporation, Tokyo (JP)

(72) Inventor: Nobuaki KAWASE, Tokyo (JP)

(73) Assignee: NEC Corporation, Tokyo (JP)

(21) Appl. No.: 18/883,436

(22) Filed: Sep. 12, 2024

Related U.S. Application Data
(63) Continuation of application No. 18/008,881, filed on Dec. 7, 2022.

(51) Int. Cl. G07C 9/25; G06V 40/16; G16H 10/60

(52) U.S. Cl. CPC G07C 9/25; G06V 40/171; G06V 40/172; G16H 10/60

(57) ABSTRACT

A passage permit device includes at least one memory storing instructions, and at least one processor. The at least one processor is configured to execute the instructions to, acquire image data including a user's face image captured by a predetermined imaging device, cause an authentication device configured to store facial feature information about the user to perform face authentication on the image data, acquire, if the face authentication is successful, proof information about the user subjected to the face authentication from a storage device configured to store the proof information related to measures taken the user to prevent from being infected, determine whether or not to permit the user to pass based on the proof information associated with the user, output a result of the determination to a predetermined terminal device corresponding to the imaging device.

This application is a Continuation of U.S. application Ser. No. 18/008,881 filed on Dec. 7, 2022, which is a National Stage Entry of PCT/JP2020/022708 filed on Jun. 9, 2020, the contents of all of which are incorporated herein by reference, in their entirety.

### TECHNICAL FIELD

The present disclosure relates to a passage permit device, system, method, and program.

### BACKGROUND ART

Knowing whether a person has effective immunity to an infectious disease is an important factor in specifying an extent to which that person's activities in his/her social live should be restricted. For this reason, means for finding out whether a person has a proof of vaccination is desired.

For example, a system described in Patent Literature 1 has a patient registration card that has been changed into an IC card, an IC card reader, and a personal computer for processing information. A patient chart number, a vaccination status, and other information items are input into the patient registration card that has been changed into an IC card.

### CITATION LIST

### Patent Literature

Patent Literature 1: Japanese Unexamined Patent Application Publication No. 2000-099589

### SUMMARY OF INVENTION

### Technical Problem

According to the above technique, by reading an IC card, it is possible to determine whether or not a user should be subject to an activity restriction. However, the technique described above requires contact with a user who may be infected with an infectious disease in order to read an IC card. Furthermore, it is impossible to determine whether or not to impose restrictions on users' activities who do not have IC cards.

The present disclosure has been made to solve such problems and an object thereof is to provide a passage permit device, system, method, and program for determining a desired passage permit while reducing contact with a person to be authenticated.

### Solution to Problem

In an example aspect of the present disclosure, a passage permit device includes image data acquisition means, authentication control means, proof information acquisition means, determination means, and output means. The image data acquisition means acquires image data including a user's face image captured by a predetermined imaging device. The authentication control means causes an authentication device configured to store facial feature information about the user to perform face authentication on the image data. The proof information acquisition means acquires, if the face authentication is successful, proof information about the user subjected to the face authentication from a storage device configured to store the proof information related to measures taken by the user to prevent from being infected (hereinafter such measures will be referred to as preventive measures). The determination means determines whether or not to permit the user to pass based on the proof information associated with the user. The output means outputs a result of the determination to a predetermined terminal device corresponding to the imaging device.

In another example aspect of the present disclosure, a computer performs a passage permit method. The computer acquires image data including a user's face image captured by a predetermined imaging device. The computer causes an authentication device configured to store facial feature information about the user to perform face authentication on the image data. The computer acquires, if the face authentication is successful, proof information about the user subjected to the face authentication from a storage device configured to store the proof information related to measures taken the user to prevent from being infected. The computer determines whether or not to permit the user to pass based on the proof information associated with the user. The computer outputs a result of the determination.

In another example aspect of the present disclosure, a program causes a computer to perform the following. The computer acquires image data including a user's face image captured by a predetermined imaging device. The computer causes an authentication device configured to store facial feature information about the user to perform face authentication on the image data. The computer acquires, if the face authentication is successful, proof information about the user subjected to the face authentication from a storage device configured to store the proof information related to measures taken the user to prevent from being infected. The computer determines whether or not to permit the user to pass based on the proof information associated with the user. The computer outputs a result of the determination.

### Advantageous Effects of Invention

According to the present disclosure, it is possible to a passage permit device, system, method, and program for determining a desired passage permit while reducing contact with a person to be authenticated.

### BRIEF DESCRIPTION OF DRAWINGS

FIG. 1 is a block diagram showing a configuration of a passage permit device according to a first example embodiment;

FIG. 2 is a flowchart showing a passage permit method according to the first example embodiment;

FIG. 3 is a flowchart showing a passage permit method according to a second example embodiment;

FIG. 4 is a block diagram showing a configuration of a passage permit device according to a third example embodiment;

FIG. 5 is a flowchart showing a passage permit method according to the third example embodiment;

FIG. 6 is a block diagram showing a configuration of a passage permission system according to a fourth example embodiment;

FIG. 7 is a block diagram showing a configuration of an authentication device;

FIG. 8 is a flowchart showing a flow of facial feature information registration processing;

FIG. 9 is a flowchart showing a flow of face authentication processing;

FIG. 10 is a block diagram showing a configuration of a terminal device;

FIG. 11 is a block diagram showing a configuration of a user terminal; and

FIG. 12 is a block diagram showing a configuration of a passage permit device; and

FIG. 13 shows an example of a screen displayed on the terminal device.

### EXAMPLE EMBODIMENT

Example embodiments of the present disclosure are described in detail below with reference to the drawings. In each drawing, the same or corresponding elements are given the same signs, and repeated descriptions are omitted as necessary for clarity.

### First Example Embodiment

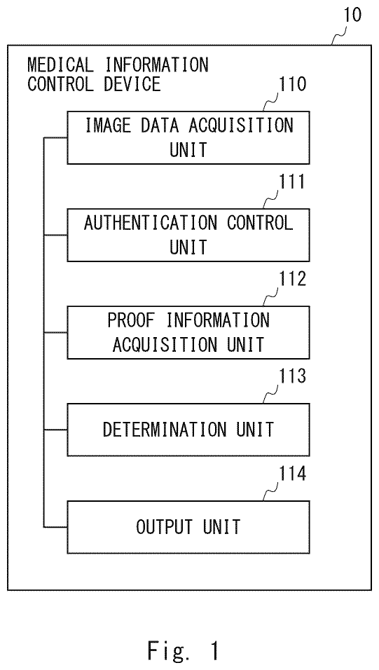

A configuration of a passage permit device is described with reference to FIG. 1. FIG. 1 is a block diagram showing a configuration of a passage permit device 10 according to a first example embodiment. The passage permit device 10 performs face authentication on a user who uses a predetermined facility to permit him/her to, for example, enter or exit the facility. Example of the predetermined facilities include commercial facilities, theaters, airports, train stations, schools, office buildings, hospitals, and immigration control facilities. The passage permit device 10 is connected to a communication network (hereinafter, the communication network is also referred to simply as a network) and performs various communications with external devices via the connected network.

More specifically, the passage permit device 10 is connected to an imaging device, an authentication device, a storage device, and a terminal device via the network. The imaging device captures the user's face and supplies image data of the captured image to the passage permit device 10. The authentication device receives the image data from the passage permit device 10 and performs face authentication on the received image data. The storage device stores the user's proof information and provides the user proof information for the face authentication to the passage permit device 10.

The terminal device receives, from the passage permit device 10, the result of the determination as to whether the user subjected to the face authentication is permitted to pass, and displays the received result of the determination. The network may be wired or wireless, or it may be any type of communication protocol. The passage permit device 10 includes an image data acquisition unit 110, an authentication control unit 111, a proof information acquisition unit 112, a determination unit 113, and an output unit 114 as main components.

The image data acquisition unit 110 acquires the image data including the user's face image captured by a predetermined imaging device. The authentication control unit 111 controls the authentication device that stores the user's facial feature information to perform face authentication on the image data. The authentication control unit 111 also receives a result of the facial recognition from the authentication device. The authentication control unit 111 receives an authentication ID of the user subjected to the authentication together with a signal indicating successful face authentication from the authentication device. When the authentication control unit 111 receives the authentication ID of the user subjected to the authentication, it supplies the received information to the proof information acquisition unit 112.

When the face authentication is successful, the proof information acquisition unit 112 acquires proof information of the user subjected to the face authentication from the storage device that stores proof information of users. More specifically, when the proof information acquisition unit 112 receives the authentication ID of the user subjected to the successful face authentication, it acquires the proof information associated with the received authentication ID. When the proof information acquisition unit 112 acquires the proof information about the user subjected to the authentication, it supplies the acquired proof information to the determination unit 113.

The proof information about the user indicates that the user is taking measures to prevent from being infected with a predetermined infectious disease. More specifically, the proof information includes, for example, information indicating that the user has an antibody against a predetermined infectious disease (antibody proof), information indicating that the user has been vaccinated against a predetermined infectious disease (vaccination proof), and so on. The proof information may also include information indicating that the user does not have an antibody against a predetermined infectious disease or that the user has not been vaccinated against a predetermined infectious disease. The proof information of the user may include profile information such as the user's name, nationality, gender, and address. The proof information of the user is registered, for example, by a medical institution that conducts antibody tests and vaccinations. The user's proof information may be registered by the user himself/herself.

In place of the above information, the proof information may include, for example, a negative test result proof for a predetermined virus as preventive measures taken by the user. The negative test result proof is information that proves, for example, that a Polymerase Chain Reaction (PCR) test is negative.

The proof information may also include future activity plan information (hereafter, future activity plan information may be referred to simply as activity plan information) in addition to the antibody proof, the vaccination proof, or the negative test result proof as the preventive measures taken by the user. The activity plan information includes a travel destination, an accommodation, stay and visit destinations planned by the user for a preset period.

The determination unit 113 determines whether or not to permit the user to pass based on the proof information associated with the user subjected to the authentication. For example, if the proof information includes the antibody proof, the determination unit 113 determines that the user is permitted to pass. On the other hand, for example, if the proof information does not include the antibody proof, the determination unit 113 determines that the user is not permitted to pass. The determination unit 113 may also determine that the user is permitted to pass when the proof information includes negative test result information. The determination unit 113 may determine that the user is permitted to pass when the activity plan information is included in the proof information.

The determination unit 113 determines that the user is permitted to pass based on the above proof information according to preset determination criteria. The preset determination criteria are set in accordance with the predetermined quarantine measures. Therefore, for example, if the activity plan of the user included in the activity plan information does not satisfy the above determination criteria, the determination unit 113 does not permit the user to pass. The determination unit 113 supplies the result of the determination to the output unit 114.

The output unit 114 outputs the result of the determination received from the determination unit 113 to a predetermined terminal device corresponding to the above imaging device. A terminal device corresponding to an imaging device means, for example, that a terminal device is integrated with an imaging device. Alternatively, a terminal device corresponding to an imaging device means, for example, when the imaging device and the terminal device are installed in the same facility, when an administrator who manages the imaging device and the terminal device is the same, or when an administrator who manages the imaging device and an administrator who manages the terminal device work together to perform a predetermined work assignment.

The imaging device and the terminal device may be mobile terminals (user terminals) owned by the user. In this case, the user terminal is, for example, a mobile phone, a portable PC, a tablet terminal, or a smartphone. In this case, the user captures his/her own face image by using the user terminal. The user terminal supplies image data to the passage permit device 10. The passage permit device 10 then receives the image data supplied from the user terminal, and upon a success of authentication, determines whether or not to permit the user to pass, and outputs the result of the determination to the user terminal. Similarly, either the imaging device or the terminal device may be a user terminal.

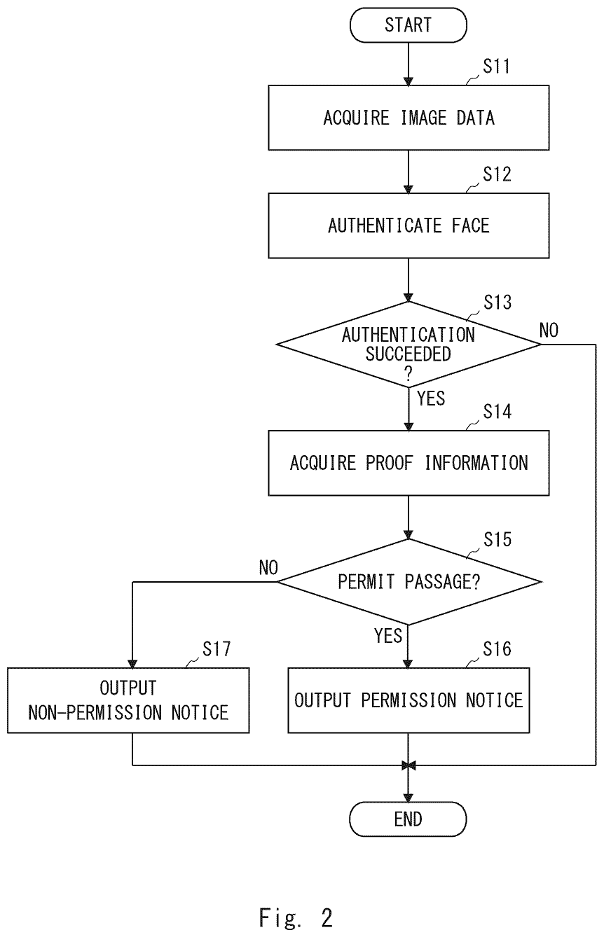

Next, the processing performed by the passage permit device 10 will be described with reference to FIG. 2. FIG. 2 is a flowchart showing a passage permit method performed by the passage permit device 10 according to the first example embodiment.

First, the passage permit device 10 acquires image data from the imaging device via the network (Step S11). In this case, the passage permit device 10 may request the imaging device to transmit the image data or may accept the transmission of image data from the imaging device at predetermined intervals.

Next, the passage permit device 10 causes the authentication device to perform face authentication (Step S12). More specifically, the passage permit device 10 supplies the image data received from the imaging device to the authentication device and requests the authentication device to perform authentication. The authentication device associates and stores the identification information of a plurality of users with the facial feature information. The authentication device extracts, for example, a predetermined feature quantity from the face image of the user included in the image data and associates the extracted feature quantity with the identification information of the user. When the authentication is successful, the authentication device also supplies the identification information of the user associated with the received image data to the passage permit device 10.

Next, the passage permit device 10 determines whether the authentication is successful (Step S13). The passage permit device 10 determines whether or not the authentication is successful, for example, by a signal sent from the authentication device. For example, if a signal indicating successful authentication is not obtained from the authentication device within a predetermined period after the transmission of a request signal for performing authentication, the passage permit device 10 does not determine that authentication is successful (Step S13: NO). In this case, the passage permit device 10 terminates the processing.

On the other hand, if, for example, a signal indicating successful authentication is obtained from the authentication device within a predetermined period after the transmission of the request signal for performing authentication, the passage permit device 10 determines that the authentication is successful (Step S13: YES). In this case, the passage permit device 10 acquires the proof information of the user subjected to the authentication that has been successful from the storage device (Step S14).

Next, the passage permit device 10 determines whether or not to permit the user subjected to the authentication to pass based on the acquired proof information (Step S15). When it is determined that the user subjected to the authentication is permitted to pass (Step S15: YES), the passage permit device 10 outputs a result of the determination (permission notice) indicating that the user is permitted pass to the terminal device (Step S16). After the passage permit device 10 outputs the result of the determination, it ends the processing.

On the other hand, when it is not determined that the user subjected to the authentication is permitted to pass (Step S15: NO), the passage permit device 10 outputs the result of the determination (notice of non-permission) to the terminal device indicating that the user is not permitted to pass (Step S17). After the passage permit device 10 outputs the result of the determination, it ends the processing.

The passage permit device 10 according to the first example embodiment has been described above. Although not included in the above processing, the passage permit device 10 may supply the proof information of the user who has succeeded in face authentication to a predetermined management device (not shown). The predetermined management device is owned by a management organization or the like that takes preventive measures using the proof information of the user. Here, a management organization or the like is, for example, a public organization that operates infrastructure, a government organization such as a municipality, or an employer who uses users as employees, and who has the authority to manage or monitor activities of the users.

Upon receiving the information from the passage permit device 10, the management organization or the like may obtain, for example, the user's face photo, antibody proof, vaccination proof, negative test result proof, activity plan information, or contact information, which are included in the proof information of the user provided from the passage permit device 10. Alternatively, the management organization or the like may be able to associate the authentication information of the user included in the proof information received from the passage permit device 10 with attribute information of the user held by the management organization or the like.

With the above configuration, the management organization or the like can contact the user by using the user's contact information (phone number, email address, social network account, etc.) provided by the passage permit device 10. Thus, if, for example, a person who may have come into contact with the user temporarily develops an infectious disease, the user can be notified or contacted. In addition, as part of the preventive measures, the management organization or the like can carry out activities to restrict activities of the users based on the activity plan and negative test result proof provided by the passage permit device 10. For example, a user may have been a close contact person if it is determined that a person affected by an infectious disease or a person infected with an infectious disease is present at a time and place included in the user's activity plan. In that case, the management organization or the like can then make recommendations to the user about controlling activities and conducting a test for the infectious disease. Alternatively, in a case where the management organization or the like can inquire whether the actual activity of the user is consistent with the activity plan, if the actual activity of the user differs from the activity plan, the management organization or the like can issue a warning to the user to control his/her activities. Note that examples of the case where the management organization or the like can inquire the actual activity of the user are cases in which the management organization or the like can capture location information of the user, or cases in which the user can acquire information such as checkpoints that the user passes at a specified date and time. Here, “if the actual activity of the user differs from the activity plan” includes the case in which the activity of the user can no longer be captured.

The passage permit device 10 includes a processor and a storage device as a configuration not shown. The storage device included in the passage permit device 10 includes a storage device such as a non-volatile memory, for example, a flash memory and an SSD (Solid State Drive). In this case, the storage device included in the passage permit device 10 stores the proof information, and the proof information of the user subjected to the successful face authentication may be read from such storage device.

A computer program (hereafter also referred to simply as a program) for executing the passage permit method according to this example embodiment is stored in the storage device of the passage permit device 10. The processor also reads the computer program from the storage device into the memory and executes the program.

Each component included in the passage permit device 10 may be implemented by dedicated hardware. Further, some or all of the constituent elements of each device may be implemented by general-purpose or dedicated circuitry, processors, etc., or a combination thereof. These constituent elements may be composed of a single chip or a plurality of chips connected via a bus. Some or all of the constituent elements of each device may be implemented by a combination of the circuitry, the program, and the like described above. The processor may be a CPU (Central Processing Unit), a GPU (Graphics Processing Unit), an FPGA (field-programmable gate array), or the like.

Further, when some or all of the constituent elements of the passage permit device 10 are implemented by a plurality of information processing apparatuses, circuitry, etc., the plurality of information processing apparatuses, circuitry, and the like, may be collectively arranged or arranged separate from each other. For example, the information processing apparatus, the circuitry, and the like may be implemented as a form where they are connected to each other via a communication network, such as a client server system, a cloud computing system, and the like. Further, the function of the passage permit device 10 may be provided in a Saas (Software as a Service) format.

The above passage permit device 10 performs face authentication of the user, and outputs the result of the determination as to whether the user is permitted to pass to the terminal device. Thus, the user using a predetermined facility can receive a determination about the permission of passage without contacting equipment, staff, and the like of such facility. On the other hand, a facility that uses the passage permit device 10 can determine whether to permit the user to pass while reducing the risk of spread of infection. That is, according to this example embodiment, the passage permit device, system, method, and program for determining a desired passage permission while reducing contact with a person to be authenticated can be provided.

### Second Example Embodiment

Next, a second example embodiment will be described. A passage permit device 10 according to the second example embodiment differs from the passage permit device according to the first example embodiment in that the passage permit device 10 according to the second example embodiment also acquires effective period information about an antibody proof or a vaccination proof.

The functional configuration of the passage permit device 10 according to this example embodiment is the same as that shown in the block diagram of FIG. 1 according to the first example embodiment. However, the proof information acquisition unit 112, the determination unit 113, and output unit 114 have the following additional functions.

The proof information acquisition unit 112 also acquires effective period information about an antibody proof or a vaccination proof. The proof information acquisition unit 112 supplies the acquired effective period information to the determination unit 113. The determination unit 113 determines whether or not to permit the user to pass based on the acquired proof information. At this time, the determination unit 113 determines whether or not to permit the user to pass in consideration of the effective period information. The determination unit 113 also determines that a warning that an effective period is shorter than a preset period is sent when the effective period is shorter than the preset period. The output unit 114 then outputs the warning to the terminal along with the result of the determination. At this time, the output unit 114 may output a warning urging vaccination in view of the warning.

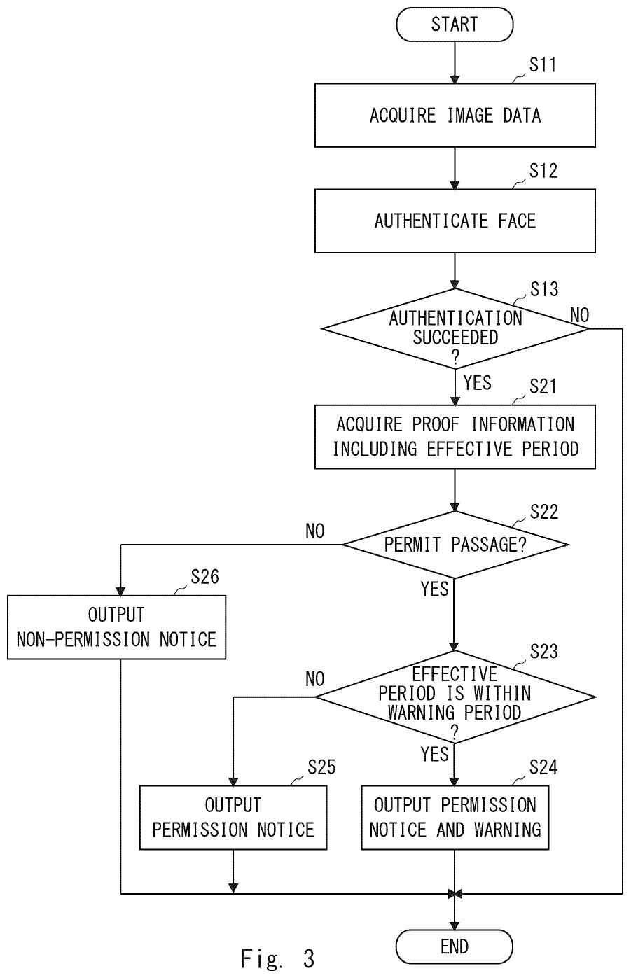

The processing performed by the passage permit device 10 according to this example embodiment will be described with reference to FIG. 3. FIG. 3 is a flowchart showing the passage permit method performed by the passage permit device 10 according to the second example embodiment. The flowchart shown in FIG. 3 differs from the flowchart shown in FIG. 2 in the processing after Step S13.

When it is determined in Step S13 that the authentication is successful (Step S13: YES), the passage permit device 10 acquires, from the storage device, the proof information of the user subjected to the authentication that has been successful (Step S21). At this time, the proof information includes the effective period information. The effective period information indicates, for example, the effective period of the antibody possessed by the user or the vaccine the user has received. The effective period of the user's antibody or vaccination is set according to individual circumstances. The individual circumstances include, for example, the user's age, the type of virus that the antibody or vaccine is targeting, and the type of vaccine. The effective period can be set when the user receives an antibody test or vaccination at a medical institution or the like.

Next, the passage permit device 10 determines whether or not to permit the user to pass who has been subjected to authentication based on the acquired proof information (Step S22). Here, the passage permit device 10 determines whether or not the proof information includes information that the person has a predetermined antibody, and also determines whether or not the effective period of the antibody has expired.

For example, if the proof information includes the predetermined antibody proof or a predetermined vaccination proof and the effective period of the antibody or vaccine has not expired, the passage permit device 10 determines that the user is permitted to pass. For example, assume that the user had an antibody with an effective period of 12 month nine months ago. In this case, the effective period of the antibody possessed by the user at the time of executing the flowchart shown in the drawing is three months. Therefore, the passage permit device 10 determines that the user is permitted to pass. In this case (Step S22: YES), the passage permit device 10 proceeds to Step S23.

On the other hand, if the proof information does not include the predetermined antibody proof or the predetermined vaccination proof, or if the effective period of the antibody or vaccine has expired, the passage permit device 10 does not determine that the user is permitted to pass. For example, suppose that a user had an antibody with an effective period of 12 months 13 months ago. In this case, the effective period of the antibody at the time of executing the flowchart shown in the drawing has already expired. Thus, the passage permit device 10 does not determine that the user is permitted to pass. In this case (Step S22: NO), the passage permit device 10 outputs a result of the determination (notice of non-permission) to the terminal device indicating that the user is not permitted to pass (Step S26). After the passage permit device 10 outputs the result of the determination, it ends the processing.

In Step S23, the passage permit device 10 determines whether the effective period is within a preset warning period (Step S23). Here, the warning period is a period shorter than the effective period and set to warn the user that the effective period is about to expire. When it is determined that the effective period included in the proof information is within the warning period (Step S23: YES), the passage permit device 10 outputs a warning to the terminal device (Step S24) in addition to a permission notice and then terminates the processing. At this time, the passage permit device 10 outputs, for example, a warning notifying the user that the expiration of the effective period is imminent. In addition, the passage permit device 10 may output, for example, a message urging the user to receive a new vaccine. When the passage permit device 10 urges the user to receive a new vaccine, the passage permit device 10 may present the type of vaccine associated with the above warning. The passage permit device 10 may also suggest one or more types of vaccine for the user to receive based on the profile information related to the vaccination of the user. The profile information related to the vaccination of the user may include, for example, the user's vaccination history, the user's age, gender, nationality, etc. The profile information related to the vaccination of the user may be included in advance in the proof information. Note that the above warning may be sent also to the management organization or the like described in the first example embodiment. On the other hand, if it is not determined that the effective period included in the proof information is within the warning period (Step S23: NO), the passage permit device 10 outputs a permission notice (Step S25) and terminates the processing.

The second example embodiment has been described above. According to the second example embodiment, a passage permit device, system, method, and program for determining a desired passage permission while reducing contact with a person to be authenticated can be provided. In addition, according to the second example embodiment, a passage permit device, system, method, and program for warning a user that an effective period of an antibody proof or a vaccination proof is approaching and reducing the spread of infectious diseases.

### Third Example Embodiment

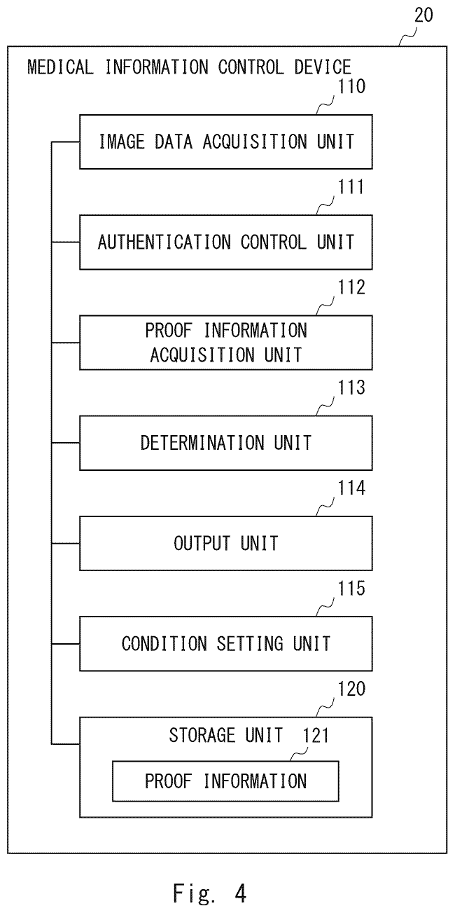

Next, a third example embodiment will be described. A permit device according to the third example embodiment differs from the passage permit devices described above in that the passage permit device according to the third example embodiment further includes a condition setting unit and a storage unit. A passage permit device 20 shown in FIG. 4 includes an image data acquisition unit 110, an authentication control unit 111, a proof information acquisition unit 112, a determination unit 113, an output unit 114, a condition setting unit 115, and a storage unit 120 as main components.

The condition setting unit 115 receives conditions for determining a passage permission from the terminal device. For example, when the passage permit device 20 is used at a departure control facility, the condition setting unit 115 accepts setting of conditions for determining a passage permission according to the destination of the user. The determination unit 113 determines whether or not to permit the user to pass based on the conditions set as described above and the proof information.

Here, an example of the conditions accepted by the condition setting unit 115 will be described. For example, suppose that a country A requires foreigners entering the country A to have an antibody A effective for three months or longer. In such a case, the condition setting unit 115 can set the condition that the effective period of the antibody A must be three months or longer for a user who departs from a country for the purpose of heading to the country A as a condition for determining a passage permission. Suppose also that a country B requires foreigners entering the country B to have a vaccination proof of a vaccine X. In such a case, the condition setting unit 115 can set the condition that a user who departs from a country for the purpose of heading to the country B must have a vaccination proof of the vaccine X as a condition for determining the passage permission.

The storage unit 120 is a storage device including a non-volatile memory and stores at least the proof information 121 of the user. In addition to the proof information 121 being associated with the identification information of the user, the proof information 121 may include index information accompanying each piece of the proof information. The index information is additional information for enabling a plurality of pieces of the proof information to be stratified, extracted, or searched.

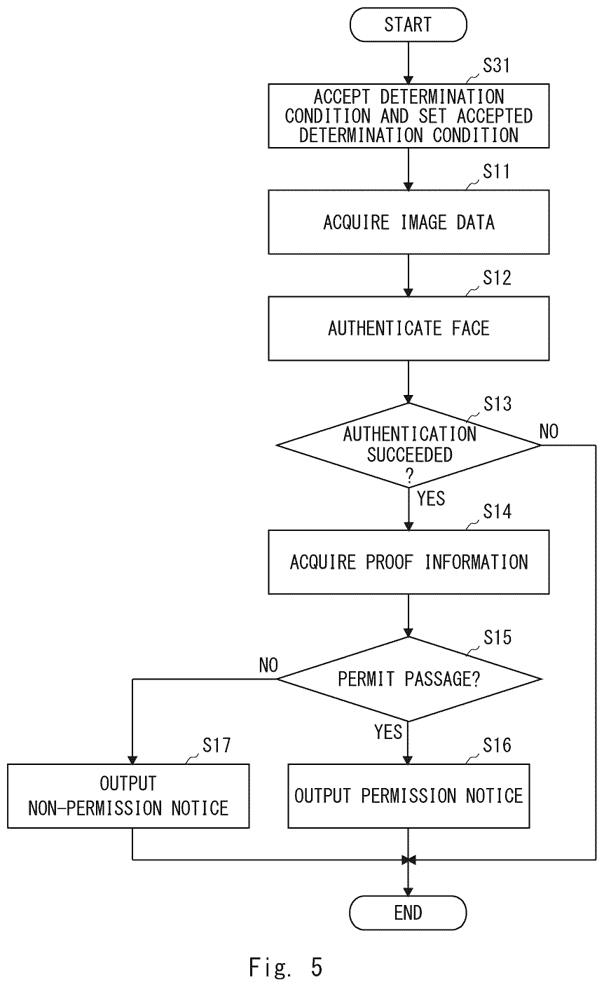

FIG. 5 is a flowchart showing a passage permit method according to the third example embodiment. The flowchart shown in FIG. 5 differs from the flowchart of FIG. 2 according to the first embodiment in that it has Step S31 before Step S11.

In the flowchart shown in FIG. 5, the condition setting unit 115 of the passage permit device 20 accepts the determination condition, and sets the accepted determination condition (Step S31). When the condition setting unit 115 sets the determination condition, the passage permit device 20 proceeds to Step S11 to acquire image data from the imaging device. The subsequent processing is similar to that in the flowchart shown in FIG. 2. However, the determination condition set in Step S31 is used when it is determined as to whether or not to permit the user to pass in Step S15. In setting the determination condition, the condition setting unit 115 may accept the condition, for example, “a determination is made according to the user's nationality”. In this case, the passage permit device 20 includes the user's nationality in the proof information. The passage permit device 20 also stores determination conditions for each user's nationality. Thus, when the face authentication of the user is successful, the passage permit device 20 can acquire information about the user's nationality and set the determination condition for the country of the acquired user's nationality. In this case, the determination condition for each nationality stored in the passage permit device 20 may be stored in advance or, for example, acquired from a computer (not shown) connected via a network.

The third example embodiment has been described above. Note that it is not necessary to perform Step S31 of the flowchart shown in FIG. 5 each time the user's face is authenticated and a permission is determined. For example, the passage permit device 20 may maintain conditions once set until they are updated.

According to the third example embodiment, a passage permit device, system, method, and program for determining a desired passage permission while reducing contact with a person to be authenticated can be provided. In addition, according to the third example embodiment, a passage permit device, system, method, and program for determining a passage permission according to individual circumstances can be provided.

### Fourth Example Embodiment

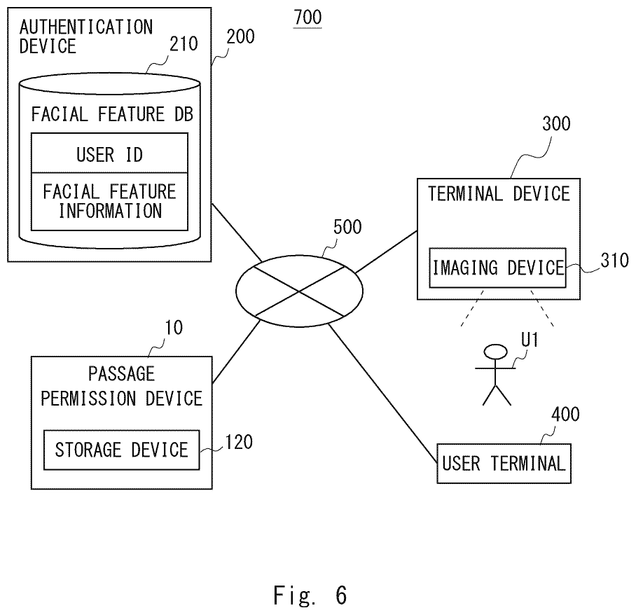

Next, a fourth example embodiment will be described. The fourth example embodiment is an example of a system that includes the passage permit device 10 according to the first example embodiment described above. FIG. 6 is a block diagram showing a configuration of a passage permission system 700 according to the fourth example embodiment. The passage permission system 700 includes the passage permit device 10, an authentication device 200, a terminal device 300, and a user terminal 400. Each of the passage permit device 10, the authentication device 200, terminal device 300, and the user terminal 400 is connected via a network 500. Note that in this example embodiment, the passage permit device 10 includes a storage unit 120. Repeated descriptions appeared in the first example embodiment are omitted as appropriate.

The passage permit device 10 is an information processing device for determining, when authentication is successful for an image in which a user U1 has been captured, a passage permission of the user U1 based on proof information of the user U1 and outputting the result of the determination to the terminal device 300. The passage permit device 10 is, for example, a server device implemented by a computer.

The authentication device 200 performs face authentication. The authentication device 200 includes a facial feature DB 210. The facial feature DB 210 is a facial feature database that stores user IDs and facial feature information of the users in association with each other. The facial feature DB 210 is an example of a facial feature information storage unit.

The terminal device 300 is an information processing device including the imaging device (imaging device 310) described in the first example embodiment. The terminal device 300 is installed in a predetermined facility that requires, for example, a passage permit. The terminal device 300 captures the user U1's face who has visited a predetermined facility, and transmits a face authentication request including the captured image to the passage permit device 10. When such face authentication is successful, the terminal device 300 also receives the result of the determination as to whether the user U1 is permitted to pass. When the terminal device 300 receives the result of the determination as to whether the user U1 is permitted to pass, it displays the result of the determination. Note that the terminal device 300 may include a driving device that opens and closes a gate according to the result of the determination. For example, when the terminal device 300 receives the result of the determination indicating that the user U1 is permitted to pass, it may cause a driving device included in the terminal device 300 to open the gate. For example, if the terminal device does not receive the result of the determination indicating that the user U1 is permitted to pass, the terminal device 300 may cause the driving device of the terminal device 300 to close the gate. A plurality of the terminal devices 300 may be arranged in a predetermined facility.

The user terminal 400 is an information processing device operated by the user U1 who intends to pass through a predetermined facility. The user terminal 400 is, for example, a communication terminal such as a smartphone, a tablet, or a PC. The user terminal may perform the same processing as or a part of the processing of the terminal device 300.

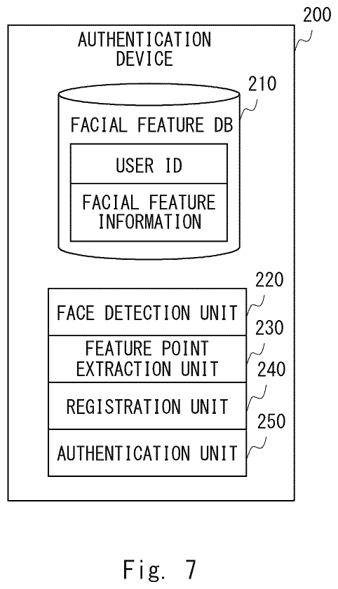

Next, a configuration of the authentication device 200 will be described in detail with reference to FIG. 7. FIG. 7 is a block diagram showing the configuration of the authentication device 200. The authentication device 200 includes a facial feature DB 210, a face detection unit 220, a feature point extraction unit 230, a registration unit 240 and an authentication unit 250.

The facial feature DB 210 is a facial feature database that stores user IDs and facial feature information of the user in association with each other. The face detection unit 220 detects a face area included in the captured image and outputs it to a feature point extraction unit 230. The feature point extraction unit 230 extracts feature points from the face area detected by the face detection unit 220 and outputs facial feature information to the registration unit 240. The facial feature information is a set of extracted feature points.

When the registration unit 240 registers the facial feature information, it issues a new user ID. The registration unit 240 registers the issued user ID and the facial feature information extracted from the registered image in the facial feature DB 210 in association with each other. The authentication unit 250 checks the facial feature information extracted from the face image with the facial feature information in the facial feature DB 210. When the two pieces of the facial feature information match, the authentication unit 250 determines that the face authentication is successful, while when the two pieces of the facial feature information do not match, it determines that the face authentication fails. The authentication unit 250 returns the success or failure of the face authentication to the passage permit device 10. Whether or not the two pieces of the facial feature information match corresponds to the success or failure of the authentication. When the face authentication is successful, the authentication unit 250 specifies the user ID associated with the successful facial feature information and returns the authentication result including the specified user ID and the fact of the successful authentication to the passage permit device 10.

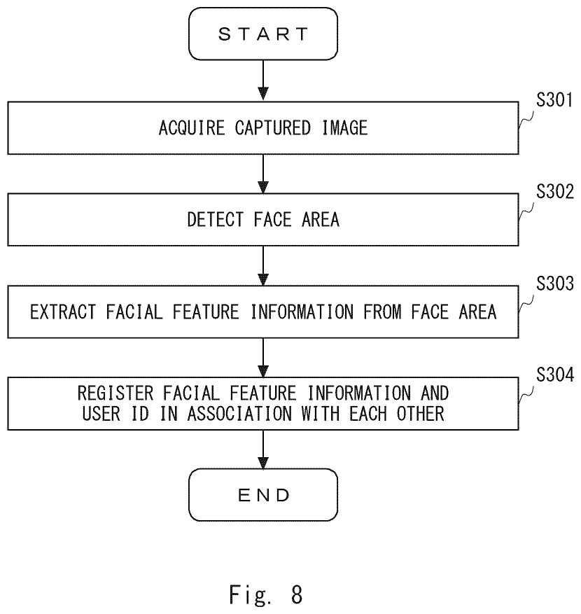

The operation of the authentication device 200 in registering the user ID and facial feature information in the facial feature DB 210 will be described with reference to FIG. 8. FIG. 8 is a flowchart showing a flow of facial feature information registration processing.

When the facial feature information is registered, a face information registration terminal (not shown) captures an image including the user's face as an image for registration image requests the authentication device 200 to register the facial feature information through the network 500. The facial information registration terminal is, for example, an information processing device such as a personal computer, a smartphone, or a tablet terminal. The face information registration terminal may be, for example, the user terminal 400. The facial feature information registration request includes the image for registration. First, the authentication device 200 acquires the image for registration included in the facial feature information registration request from the face information registration terminal via the network 500 (Step S301).

Next, the face detection unit 220 detects the face area included in the image for registration (Step S302), and outputs the detected face area to the feature point extraction unit 230. Next, the feature point extraction unit 230 extracts the feature points from the face area and outputs the facial feature information to the registration unit 240 (Step S303). Next, the registration unit 240 issues a user ID corresponding to the output facial feature information and registers the user ID and the facial feature information in the facial feature DB 210 in association with each other (Step S304).

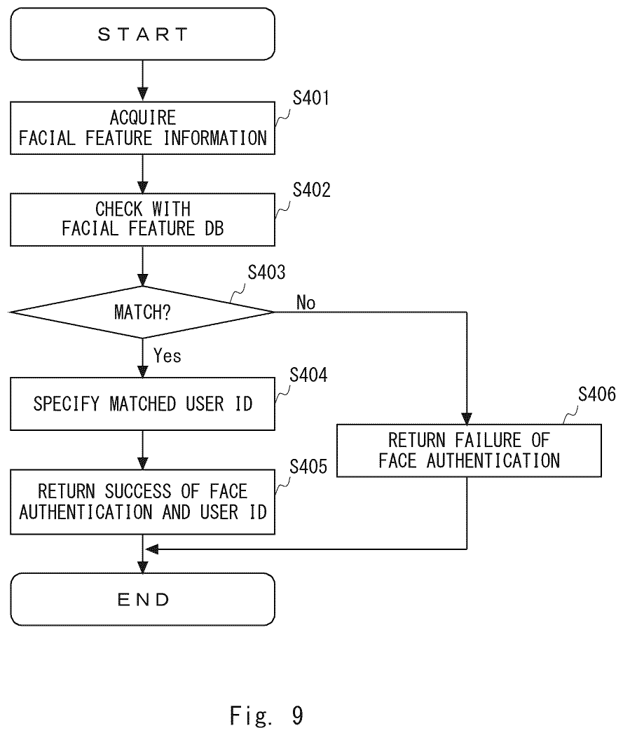

The operation of the authentication device 200 in performing face authentication will be described with reference to FIG. 9. FIG. 9 is a flowchart showing the flow of face authentication processing. When face authentication is performed, the terminal device 300 captures an image including the user's face as an image for authentication, and issues a face authentication request to the passage permit device 10 via the network 500. The face authentication request includes the image for authentication. First, the passage permit device 10 transmits the face area or facial feature information extracted from the authentication image to the authentication device 200. The feature point extraction unit 230 receives the facial feature information to thereby extract the facial feature information from the received face area or acquire the facial feature information (Step S401).

Next, the authentication unit 250 checks the acquired facial feature information with the facial feature DB 210 (Step S402). When the two pieces of the facial feature information match (Step S403: YES), the authentication unit 250 specifies the user ID of the user whose facial feature information matches with the facial feature information in the facial feature DB 210 (Step S404), and returns a success of the face authentication and the identified user ID to the passage permit device 10 (Step S405). When there is no matching facial feature information (Step S403: NO), the authentication unit 250 returns a failure of the face authentication to the passage permit device 10 (Step S406). The authentication device 200 can estimate the user's age and gender based on the facial feature information. In Step S406, assume that the authentication unit 250 sends a reply including the estimated age and gender to the passage permit device 10.

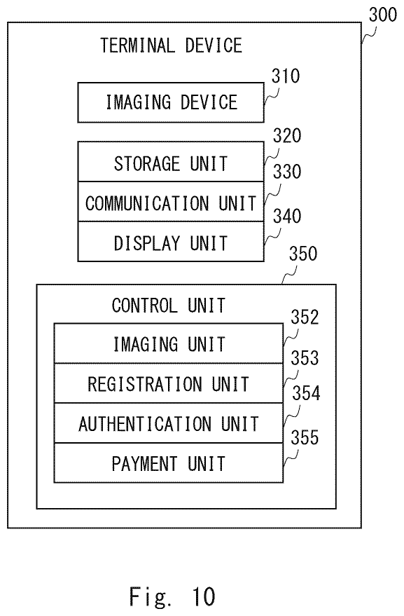

Next, the configuration of the terminal device 300 will be described in detail with reference to FIG. 10. The terminal device 300 has, as a hardware configuration, an imaging device such as a camera, a display device, and a computer. The terminal device 300 is a device that issues a face authentication request by face authentication. The terminal device 300 includes, but is not limited to, a terminal that performs entry control by face authentication, a terminal that performs payment based on face authentication, and a digital signage that presents information in response to face authentication.

FIG. 10 is a block diagram showing the configuration of the terminal device 300. The terminal device 300 has an imaging device 310, a storage unit 320, a communication unit 330, a display unit 340 and a control unit 350. The imaging device 310 captures images.

The storage unit 320 is a storage device that stores programs for implementing respective functions of the terminal device 300. The communication unit 330 is a communication interface with the network 500. The display unit 340 is a display device that displays the face authentication result, passage permission information, and the like to the user. The control unit 350 controls the hardware of the terminal device 300. The control unit 350 includes an imaging unit 352, a registration unit 353, an authentication unit 354, and a payment unit 355.

The imaging unit 352 issues an imaging request to the imaging device 310. The imaging device 310 captures an image including the user's face. The image captured by the imaging device 310 is used as the image for registration and the image for authentication. The registration unit 353 transmits a facial feature information registration request to the authentication device 200 via the network 500. The facial feature information registration request includes the image for registration captured by the imaging device 310. The authentication unit 354 transmits the face authentication request to the passage permit device 10 via the network 500. The face authentication request includes the image for authentication captured by the imaging device 310. The authentication unit 354 receives the success or failure of the face authentication from the passage permit device 10, and causes the display unit 340 to display the result.

The payment unit 355 executes payment processing upon successful face authentication. The control unit 350 may include a recording unit (not shown) instead of the payment unit 355. The recording unit may record each user's entry to and exit from the facility where the terminal device 300 is installed upon successful face authentication.

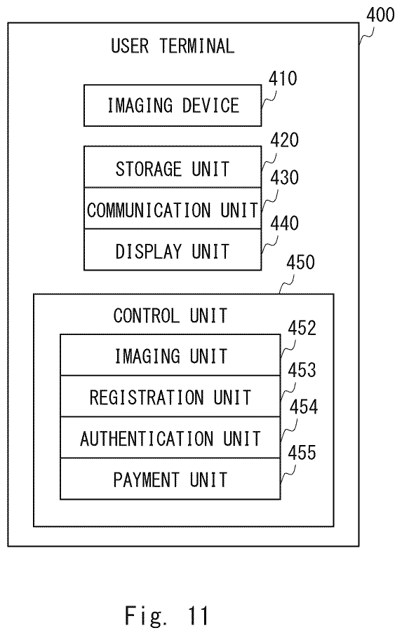

Next, a configuration of the user terminal 400 will be described in detail with reference to FIG. 11. FIG. 11 is a block diagram showing the configuration of the user terminal 400. The user terminal 400 includes an imaging device 410, a storage unit 420, a communication unit 430, a display unit 440, and a control unit 450. The control unit 450 includes an imaging unit 452, a registration unit 453, an authentication unit 454, and a payment unit 455. Since the function of each component provided in the user terminal 400 is the same as that of each component provided in the terminal device 300, descriptions thereof is omitted.

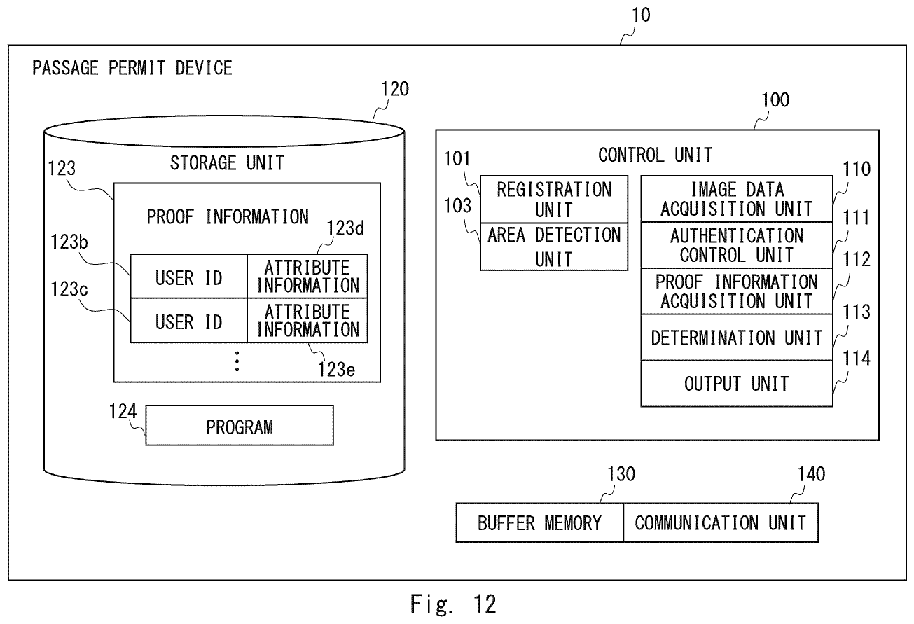

Next, the configuration of the passage permit device 10 will be described in detail with reference to FIG. 12. FIG. 12 is a block diagram showing the configuration of the passage permit device 10. The passage permit device 10 includes a control unit 100, a storage unit 120, a buffer memory 130, and a communication unit 140.

The storage unit 120 is a non-volatile storage device that stores at least proof information 123 and a program 124. The proof information 123 is a set of the user ID of each user and proof information associated with the user. The user ID is identification information that can identify the user, such as name, telephone number, email address, or any identification number. Proof information 123d and 123e are associated with user IDs 123b and 123c, respectively. That is, the storage unit 120 has identification information of a plurality of users associated with the user's proof information. The proof information 123d and 123e are, for example, the aforementioned antibody proof or vaccination proof, or the like, but the present disclosure is not limited to them. The proof information 123d and 123e may include gender, age, hobby, physical information, or the like. The program 124 is a computer program in which the passage permission processing according to this example embodiment is implemented.

The buffer memory 130 is a storage area for temporarily storing processing contents of the control unit 100, and is a volatile storage device such as RAM (Random Access Memory). The communication unit 140 is an interface for communication with the outside of the passage permit device 10.

The control unit 100 has a registration unit 101, an area detection unit 103, an image data acquisition unit 110, an authentication control unit 111, a proof information acquisition unit 112, a determination unit 113, and an output unit 114. The control unit 100 is a control device that controls the operation of the passage permit device 10, for example, a processor such as a CPU. The control unit 100 reads the program 124 from the storage unit 120 into the buffer memory 130 and executes it. By doing so, the control unit 100 implements functions as the registration unit 101, the area detection unit 103, the image data acquisition unit 110, the authentication control unit 111, the proof information acquisition unit 112, the determination unit 113, and the output unit 114.

The registration unit 101 receives a registration request from the terminal device 300 or the user terminal 400 via the network 500. The registration unit 101 issues a user ID corresponding to the user ID included in the received registration request. The registration unit 101 generates the proof information 123 by associating the issued user ID with a plurality of pieces of proof information, and registers the generated proof information 123 in the storage unit 120.

The image data acquisition unit 110 acquires image data from the imaging device. The area detection unit 103 detects the face area from the image data. For example, the area detection unit 103 analyzes the image data to recognize the face area of the user. When the face area is specified in the image data, the authentication control unit 111 causes the authentication device 200 to perform face authentication using the facial feature information for each specified face area.

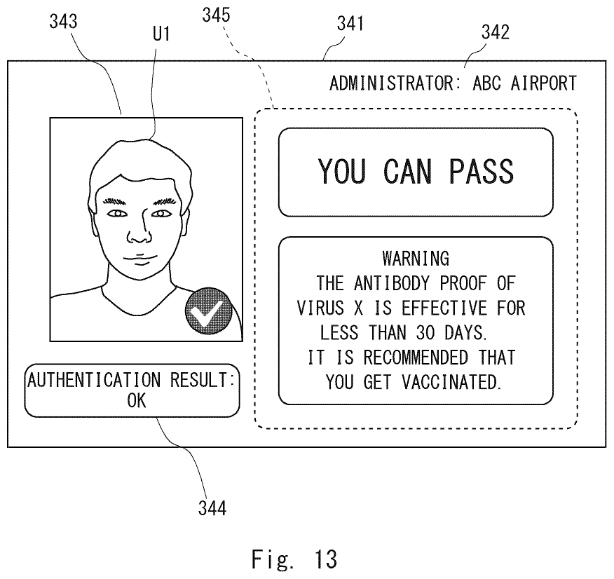

The screen displayed by the terminal device 300 will be described with reference to FIG. 13. FIG. 13 shows an example of the screen displayed on the terminal device. FIG. 13 shows a screen 341 displayed by the terminal device 300. The screen 341 includes an information display 342 about the administrator, an image 343 for face authentication of the user U1, a result display 344 for face authentication, and a determination result display 345.

In the example shown in FIG. 13, the administrator of the terminal device 300 is an airport facility named ABC Airport. At the ABC Airport, as the administrator, the passage permit device 10 is used to manage traffic at departure gates. Under these circumstances, at the ABC Airport, an operation of capturing the user U1's face, who is a passenger, by using the imaging device and requesting a result of the determination is performed. In this way, the passage permit device 10 caused the authentication device 200 to perform the authentication. Since the face authentication is successful, the passage permit device 10 has transmitted the result of the determination for the passage permission of the user U1 to the terminal device 300.

On the determination result display 345, the text “You can pass” which means a permission to pass has been given, is displayed as the result of the determination as to whether the user U1 is permitted to pass. A warning is also displayed at the bottom of the result. The warning includes the message “The antibody proof of virus X is effective for less than 30 days. It is recommended that you get vaccinated”. The display shown in FIG. 13 is an example, and other information may be displayed in addition to or in place of the information shown here. For example, when a determination condition has been set in advance in the determination of the passage permission of the user U1, the set determination condition may be displayed. If the user U1 is not permitted to pass, the reason for the disapproval may be displayed.

The passage permission system 700 according to the fourth example embodiment has been described above. The passage permission system 700 according to the fourth embodiment may include the passage permit device according to the second or third example embodiment instead of the passage permit device 10 according to the first example embodiment. In addition, the passage permission system 700 may detect the user U1's body surface temperature while performing the face authentication and set the above determination condition according to the detected body surface temperature. For example, even if the result of the determination output by the passage permit device 10 includes a content permitting the user U1 to pass, the passage permitting system 700 may have a function of not permitting the user to pass if the detected body surface temperature is higher than a predetermined threshold.

According to the fourth example embodiment, a passage permit device, system, method, and program for determining a desired passage permission while reducing contact with a person to be authenticated can be provided.

In the above example, the program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

The present disclosure is not limited to the above example embodiments and may be changed as appropriate without departing from the scope.

The whole or part of the example embodiments disclosed above can be described as, but not limited to, the following supplementary notes.

### Supplementary Note 1

A passage permit device comprising:
image data acquisition means for acquiring image data including a user's face image captured by a predetermined imaging device;
authentication control means for causing an authentication device configured to store facial feature information about the user to perform face authentication on the image data;
proof information acquisition means for acquiring, if the face authentication is successful, proof information about the user subjected to the face authentication from a storage device configured to store the proof information related to measures taken the user to prevent from being infected;
determination means for determining whether or not to permit the user to pass based on the proof information associated with the user; and
output means for outputting a result of the determination to a predetermined terminal device corresponding to the imaging device.

### Supplementary Note 2

The passage permit device according to Supplementary note 1, wherein
the proof information acquisition means also acquires effective period information about an effective period of an antibody proof or a vaccination proof included in the proof information, and
the determination means determines whether or not to permit the user to pass based on the effective period information.

### Supplementary Note 3

The passage permit device according to Supplementary note 2, wherein
the determination means determines that, if the effective period is shorter than a preset period, a warning that the effective period is shorter than the preset period is sent, and
the output means outputs the warning together with the result of the determination.

### Supplementary Note 4

The passage permit device according to Supplementary note 3, wherein
the output means outputs, as the warning, a warning urging vaccination in view of the warning.

### Supplementary Note 5

The passage permit device according to any one of Supplementary notes 1 to 4, further comprising:
a condition setting unit configured to accept a condition for the determination from the terminal device, wherein
the determination means determines whether or not to permit the user to pass based on the set condition and the proof information.

### Supplementary Note 6

The passage permit device according to any one of Supplementary notes 1 to 5, wherein
the proof information acquisition means acquires the proof information generated by a medical institution.

### Supplementary Note 7

The passage permit device according to any one of Supplementary notes 1 to 6, wherein
the proof information acquisition means acquires, in addition to the proof information, activity history information about an activity history of the user, and
the determination means makes the determination based on the activity history and the proof information.

### Supplementary Note 8

A passage permission system comprising:
the passage permit device according to any one of Supplementary notes 1 to 7; and
a storage device configured to store the user and the proof information about the user in association with each other.

### Supplementary Note 9

A passage permission system comprising:
the passage permit device according to any one of Supplementary notes 1 to 7; and an authentication device configured to receive the image data from the passage permit device and perform the face authentication.

### Supplementary Note 10

The passage permit system according to Supplementary note 8 or 9, further comprising:

a gate opening and closing device configured to receive the result of the determination from the output means, and open or close a gate located in a passage path of the user according to the result of the determination.

### Supplementary Note 11

A passage permit method performed by a computer, the passage permit method comprising:
acquiring image data including a user's face image captured by a predetermined imaging device;
causing an authentication device configured to store facial feature information about the user to perform face authentication on the image data;
acquiring, if the face authentication is successful, proof information about the user subjected to the face authentication from a storage device configured to store the proof information related to measures taken the user to prevent from being infected;
determining whether or not to permit the user to pass based on the proof information associated with the user; and
outputting a result of the determination.

### Supplementary Note 12

A non-transitory computer readable medium storing a passage permission program for causing a computer to perform:
processing of acquiring image data including a user's face image captured by a predetermined imaging device;
processing of causing an authentication device configured to store facial feature information about the user to perform face authentication on the image data;
processing of acquiring, if the face authentication is successful, proof information about the user subjected to the face authentication from a storage device configured to store the proof information related to measures taken the user to prevent from being infected;
processing of determining whether or not to permit the user to pass based on the proof information associated with the user; and
processing of outputting a result of the determination.

Note that the present disclosure is not limited to the above example embodiments and can be modified as appropriate without departing from the scope.

### REFERENCE SIGNS LIST

10 PASSAGE PERMIT DEVICE
20 PASSAGE PERMIT DEVICE
110 IMAGE DATA ACQUISITION UNIT
111 AUTHENTICATION CONTROL UNIT
112 PROOF INFORMATION ACQUISITION UNIT
113 DETERMINATION UNIT
114 OUTPUT UNIT
115 CONDITION SETTING UNIT
120 STORAGE UNIT
121 PROOF INFORMATION
130 BUFFER MEMORY
140 COMMUNICATION UNIT
200 AUTHENTICATION DEVICE
210 FACIAL FEATURE DB
220 FACE DETECTION UNIT
230 FEATURE POINT EXTRACTION UNIT
240 REGISTRATION UNIT
250 AUTHENTICATION UNIT
300 TERMINAL DEVICE
310 IMAGING DEVICE
320 STORAGE UNIT
330 COMMUNICATION UNIT
340 DISPLAY UNIT
350 CONTROL UNIT
400 USER TERMINAL
410 IMAGING DEVICE
420 STORAGE UNIT
430 COMMUNICATION UNIT
440 DISPLAY UNIT
450 CONTROL UNIT
500 NETWORK
700 PASSAGE PERMISSION SYSTEM

1. A passage permit device comprising:
at least one memory storing instructions, and
at least one processor configured to execute the instructions to;
acquire image data including a user's face image captured by a predetermined imaging device;
cause an authentication device configured to store facial feature information about the user to perform face authentication on the image data;
acquire, if the face authentication is successful, proof information about the user subjected to the face authentication from a first storage device configured to store the proof information related to measures taken the user to prevent from being infected;
acquire, if the face authentication is successful, information about a destination of the user subjected to the face authentication from a second storage device configured to store the information about the destination of the user;
accept a condition set according to the destination of the user, the condition being a condition used for determining a passage permission;
determine whether or not to permit the user to pass based on the proof information associated with the user and the set condition; and
output a result of the determination to a predetermined terminal device corresponding to the imaging device.

2. The passage permit device according to claim 1, wherein
the at least one processor is further configured to execute the instructions to
acquire effective period information about an effective period of an antibody proof or a vaccination proof included in the proof information, and
determine whether or not to permit the user to pass based on the effective period information and the set condition.

3. The passage permit device according to claim 2, wherein
the at least one processor is further configured to execute the instructions to
determine that, if the effective period is shorter than a preset period, a warning that the effective period is shorter than the preset period is sent, and
output the warning together with the result of the determination.

4. The passage permit device according to claim 3, wherein
the at least one processor is further configured to execute the instruction to
output, as the warning, a warning urging vaccination in view of the warning.

5. The passage permit device according to claim 1, wherein
the at least one processor is further configured to execute the instruction to
acquire the proof information generated by a medical institution.

6. The passage permit device according to claim 1, wherein
the at least one processor is further configured to execute the instructions to
acquire, in addition to the proof information, activity history information about an activity history of the user, and
make the determination based on the activity history, the proof information, and the set condition.

7. A passage permit method performed by a computer, the passage permit method comprising:
acquiring image data including a user's face image captured by a predetermined imaging device;
causing an authentication device configured to store facial feature information about the user to perform face authentication on the image data;
acquiring, if the face authentication is successful, proof information about the user subjected to the face authentication from a first storage device configured to store the proof information related to measures taken the user to prevent from being infected;
acquiring, if the face authentication is successful, information about a destination of the user subjected to the face authentication from a second storage device configured to store the information about the destination of the user;
accepting a condition set according to the destination of the user, the condition being a condition used for determining a passage permission;
determining whether or not to permit the user to pass based on the proof information associated with the user and the set condition; and
outputting a result of the determination to a predetermined terminal device corresponding to the imaging device.

8. The passage permit method according to claim 7, further comprising:
acquiring effective period information about an effective period of an antibody proof or a vaccination proof included in the proof information, and
determining whether or not to permit the user to pass based on the effective period information and the set condition.

9. The passage permit method according to claim 8, further comprising:
determining that, if the effective period is shorter than a preset period, a warning that the effective period is shorter than the preset period is sent, and
outputting the warning together with the result of the determination.

10. The passage permit method according to claim 9, further comprising:
outputting, as the warning, a warning urging vaccination in view of the warning.

11. The passage permit method according to claim 7, further comprising:
acquiring the proof information generated by a medical institution.

12. The passage permit method according to claim 7, further comprising:
acquiring, in addition to the proof information, activity history information about an activity history of the user, and
making the determination based on the activity history, the proof information, and the set condition.

13. A non-transitory computer readable medium storing a passage permission program for causing a computer to perform:
processing of acquiring image data including a user's face image captured by a predetermined imaging device;
processing of causing an authentication device configured to store facial feature information about the user to perform face authentication on the image data;
processing of acquiring, if the face authentication is successful, proof information about the user subjected to the face authentication from a first storage device configured to store the proof information related to measures taken the user to prevent from being infected;
processing of acquiring, if the face authentication is successful, information about a destination of the user subjected to the face authentication from a second storage device configured to store the information about the destination of the user;
processing of accepting a condition set according to the destination of the user, the condition being a condition used for determining a passage permission;
processing of determining whether or not to permit the user to pass based on the proof information associated with the user and the set condition; and
processing of outputting a result of the determination to a predetermined terminal device corresponding to the imaging device.

14. The non-transitory computer readable medium according to claim 13, wherein the passage permission program further causes the computer to perform:
processing of acquiring effective period information about an effective period of an antibody proof or a vaccination proof included in the proof information, and
processing of determining whether or not to permit the user to pass based on the effective period information and the set condition.

15. The non-transitory computer readable medium according to claim 14, wherein the passage permission program further causes the computer to perform:
processing of determining that, if the effective period is shorter than a preset period, a warning that the effective period is shorter than the preset period is sent, and
processing of outputting the warning together with the result of the determination.

16. The non-transitory computer readable medium according to claim 15, wherein the passage permission program further causes the computer to perform processing of outputting, as the warning, a warning urging vaccination in view of the warning.

17. The non-transitory computer readable medium according to claim 13, wherein the passage permission program further causes the computer to perform processing of acquiring the proof information generated by a medical institution.

18. The non-transitory computer readable medium according to claim 13, wherein the passage permission program further causes the computer to perform:
processing of acquiring, in addition to the proof information, activity history information about an activity history of the user, and
processing of making the determination based on the activity history, the proof information, and the set condition.
